(19) United States
(12) Patent Application Publication
(10) Pub. No.: US 2010/0261805 A1
(43) Pub. Date: Oct. 14, 2010

(54) OLIGOMERIC POLYOLS FROM PALM-BASED OILS AND POLYURETHANE COMPOSITIONS MADE THEREFROM

(76) Inventors: Timothy W. Abraham, Minnetonka, MN (US); Jack A. Carter, Greensboro, NC (US); Jeff Malsam, Minneapolis, MN (US); Alisa B. Zlatanic, Pittsburg, KS (US)

Correspondence Address: CARGILL, INCORPORATED, P.O. Box 5624, MINNEAPOLIS, MN, 55440-5624, US

(21) Appl. No.: 12/225,650

(22) PCT Filed: Mar. 27, 2007
(86) PCT No.: PCT/US07/07657

§ 371 Date: Jun. 22, 2010

Related U.S. Application Data
(60) Provisional application No. 60/786,594, filed on Mar. 27, 2006.

(51) Int. Cl. C08G 18/32; C07C 29/151

(52) U.S. Cl. 521/170; 568/867; 252/182.24

(57) ABSTRACT

The invention relates to oligomeric polyols that are made from palm-based oils and to polyurethane compositions comprising the oligomeric palm-based polyols. Oligomeric polyols of the invention comprise a ring-opened and oligomerized epoxidized palm-based oil composition comprising about 40% wt. or greater oligomers, a hydroxyl number of about 65 or less, a number average hydroxyl functionality (fₙ) of about 2.5 or less, and a viscosity at 25° C. of about 4 Pa-s or less.

### CROSS-REFERENCE TO RELATED APPLICATIONS

This application claims the benefit of U.S. Provisional Application having Ser. No. 60/786,594, filed Mar. 27, 2006, entitled “OLIGOMERIC POLYOLS FROM PALM-BASED OILS AND POLYURETHANE COMPOSITIONS MADE THEREFROM”, the disclosure of which is incorporated herein by reference.

### BACKGROUND

Petroleum-derived polyols have been widely used in the manufacturing of polyurethane foams. Recently, however, there has been an increased interest in the use of renewable resources in the manufacturing of polyurethane foams. This has led to research into developing vegetable oil-based polyols that are suitable as replacements for petroleum-derived polyols in polyurethane foams.

One method of making a polyol from a vegetable oil is to epoxidize an unsaturated vegetable oil, followed by ring-opening of at least a portion of the epoxide groups to form alcohol groups. The usefulness of the resulting polyol in polyurethane foam applications depends upon the properties of the resulting polyol. Examples of properties that may be important include the number average hydroxyl functionality (fₙ), hydroxy number (OH number), molecular weight (e.g., Mₙ and M_{w}), and the viscosity. For flexible slabstock polyurethane foams in particular, vegetable oil-based polyols that have a low hydroxyl functionality (e.g., less than about 2.5), low OH number (less than about 65), and low viscosity are desirable as they may be used as replacements for petroleum-derived triols, in whole or in part, in flexible slabstock foam formulations.

In view of the foregoing, what is desired is an oligomeric vegetable oil-based polyol that has properties suitable for use in polyurethane foams, in particular, flexible slabstock polyurethane foams.

### SUMMARY

The invention relates to oligomeric polyols that are made from palm-based oils and to polyurethane compositions comprising the oligomeric palm-based polyols.

In one aspect, the invention provides oligomeric polyols that are made from palm-based oils. In embodiments of the invention, the oligomeric polyol comprises a ring-opened and oligomerized epoxidized palm-based oil composition comprising about 40% wt. or greater oligomers, a hydroxyl number of about 65 or less, a number average hydroxyl functionality (fₙ) of about 2.5 or less, and a viscosity at 25° C. of about 4 Pa·s or less.

Oligomeric polyols of the invention can be synthesized by first epoxidizing at least a portion of the double bonds that are present in the palm-based oil composition. The epoxidized palm-based oil composition is then ring-opened and oligomerized to form an oligomeric polyol of the invention. The ring-opening reaction is typically conducted in the presence of a ring-opener and a ring-opening catalyst. Examples of ring-openers include water, monofunctional alcohol (e.g., methanol and ethanol) and polyols (e.g., ethylene glycol). An exemplary ring-opening catalyst is hydrofluoroboric acid (HBF₄). Examples of palm-based oils include palm oil, palm olein (including palm super olein), palm stearin, palm kernel oil, palm kernel olein, or palm kernel stearin.

In some embodiments, the oligomeric polyols of the invention have a degree of ring opening and oligomerization that provide a desired balance of properties. For example, the degree of ring-opening and oligomerization can be controlled to provide an oligomeric polyol having a desired hydroxyl (OH) number, hydroxyl functionality, residual epoxy oxygen content (EOC), viscosity, molecular weight, and the like. For example, in some embodiments, the oligomeric polyol has a hydroxyl number (OH number) of about 65 or less, for example, about 40 to about 65. In some embodiments, the oligomeric polyol has a residual epoxy oxygen content (EOC) of about 0.2% to about 2.5%. In some embodiments, the oligomeric polyol has about 40% or greater oligomers, for example, about 50% or greater oligomers or about 60% or greater oligomers. In some embodiments, the oligomeric polyol has a viscosity of about 4 Pa·s or less. In some embodiments, the oligomeric polyol has a number average hydroxyl functionality (fₙ) of about 2.5 or less.

In exemplary embodiments, the oligomeric polyols are made by ring-opening a composition comprising epoxidized palm olein. Palm olein is the liquid fraction that is obtained from palm oil by fractionation after crystallization at a controlled temperature. As compared to palm oil, palm olein is higher in unsaturated fatty acids, has a higher iodine value (IV), and has a lower melting point. In some embodiments, palm olein has more unsaturated fatty acids than saturated fatty acids. Palm olein typically has an iodine value (IV) of about 55 or greater, for example, from about 55 to about 65. Included within palm olein is palm super olein, which is prepared by fractionation of palm olein. The additional fractionation raises the unsaturated fatty acid content and iodine value (IV) of palm super olein as compared to palm olein.

In some embodiments, the epoxidized palm-based oil composition comprises one or more epoxidized non-palm based oils. Examples on non-palm-based oils include epoxidized vegetable oils, for example, fully- or partially-epoxidized soybean oil. In an exemplary embodiment, the palm-based oil composition comprises a mixture of epoxidized palm olein and epoxidized soybean oil.

Oligomeric polyols made from epoxidized palm olein have a desirable balance of high oligomer content and low viscosity. In some embodiments, the oligomeric polyols made from epoxidized palm olein have a viscosity of about 4 Pa·s or less, for example, about 3 Pa·s or less, about 2 Pa·s or less, about 1 Pa·s or less, or about 0.7 Pa·s or less. In some embodiments, the oligomeric polyols made from epoxidized palm olein have viscosities ranging from about 0.5 Pa·s to about 2 Pa·s. In some embodiments, the oligomeric polyols made from epoxidized palm olein have about 40% or greater oligomers (e.g., about 50% or greater, about 55% or greater, about 60% or greater, or about 65% or greater) and have viscosities of about 4 Pa·s or less. In some embodiments, the oligomeric polyols made from epoxidized palm olein have about 50% to about 70% oligomers and have viscosities ranging from about 0.5 Pa·s to about 2 Pa·s.

In another aspect, the invention provides polyurethane compositions comprising the oligomeric polyols of the invention. In some embodiments, the polyurethane compositions comprise: (a) a polyisocyanate; and (b) an isocyanate-reactive composition comprising an oligomeric polyol of the invention. In some embodiments, the polyurethane compositions are suitable for use in polyurethane foams, for example, flexible slabstock polyurethane foams.

In some embodiments, the oligomeric polyols are used as partial or full replacements for petroleum-derived polyols in flexible slabstock polyurethane foam formulations. For example, the oligomeric polyols can replace at least a portion of a petroleum-derived triol, for example, a petroleum-derived triol having a molecular weight of about 3000 Da and an OH value of about 56. Accordingly, in some embodiments, the isocyanate-reactive composition comprises an oligomeric polyol and a petroleum-derived polyol. Typically, the polyurethane-reactive composition comprises about 10% wt. to about 60% wt. oligomeric polyol and about 40% wt. to about 90% wt. petroleum-derived triol, or about 15% wt. to about 40% wt. oligomeric polyol and about 60% wt. to about 85% wt. petroleum-derived polyol.

As used herein “polyol” refers to a molecule having an average of greater than 1.0 hydroxyl groups per molecule. It may also include other functionalities.

As used herein “epoxidized palm-based oil composition” refers to a composition comprising one or more fully-epoxidized palm-based oils, one or more partially-epoxidized palm-based oils, or a mixture thereof. Epoxidized palm-based oil compositions may optionally comprise one or more fully- or partially-epoxidized non-palm-based oils.

As used herein “oligomeric palm-based polyol” or “oligomeric polyol” refers to a non-naturally occurring polyol prepared by ring-opening and oligomerizing an epoxidized palm-based oil composition.

As used herein “flexible slabstock polyurethane foam” refers to flexible polyurethane foam that is made in the form of a long block or bun of nominal rectangular cross-section. Some types of flexible slabstock polyurethane foams include conventional foams, high-resilience foams, filled foams, and high load-bearing foams.

As used herein “hydroxyl number” indicates the number of reactive hydroxyl groups available for reaction. It is expressed as the number of milligrams of potassium hydroxide equivalent to the hydroxyl content of one gram of the sample.

As used herein “isocyanate-reactive composition” refers to a composition that includes reactants having functional groups that are capable of reacting with isocyanate groups. Examples of isocyanate-reactive functional groups include alcohols (e.g., polyols) and amines (e.g., polyamines).

As used herein “petroleum-derived polyol” refers to a polyol manufactured from a petroleum feedstock.

### BRIEF DESCRIPTION OF THE FIGURES

The present invention will be further explained with reference to the appended FIGS., wherein:

FIG. 1 is GPC chromatogram traces of polyol 1-A (—) and oligomeric soybean oil based polyol of the type reported in WO 2006/012344A1 (— —); and

FIG. 2 is GPC chromatogram traces of polyols 1-A (—) 1-B (— —), 1-C (— • • —), DURKEX NT100 palm olein (. . .) and epoxidized DURKEX NT100 palm olein (- - -).

### DETAILED DESCRIPTION

The invention relates to oligomeric polyols that are prepared from palm-based oils and to polyurethane compositions comprising the oligomeric polyols.

In some embodiments, the oligomeric polyol comprises about 40% wt. or greater oligomers, a hydroxyl number of about 65 or less, a number average hydroxyl functionality of about 2.5 or less, and a viscosity at 25° C. of about 4 Pa·s or less. Oligomeric polyols of the invention may be prepared, for example, by the process of
(a) providing a palm-based oil comprising fatty-acid triglycerides comprising carbon-carbon double bonds;
(b) epoxidizing at least a portion of the carbon-carbon double bonds present in the palm-based oil to form an epoxidized palm-based oil; and
(c) ring-opening and oligomerizing the epoxidized palm-based oil to form the oligomeric polyol.

### Palm-Based Oils:

As used herein, the term “palm-based oil” refers to an oil or oil fraction obtained from the mesocarp and/or kernel of the fruit of the oil palm tree. Palm-based oils include palm oil, palm olein, palm stearin, palm kernel oil, palm kernel olein, palm kernel stearin, and mixtures thereof. Palm-based oils may be crude, refined, degummed, bleached, deodorized, fractionated, or crystallized. In many embodiments, the palm-based oils are refined, bleached, and deodorized (i.e., an “RBD” oil).

Palm oil refers to the oil derived from the mesocarp of the oil palm fruit. Palm oil is typically a semi-solid at room temperature and comprises about 50% saturated fatty acids and about 50% unsaturated fatty acids. Palm oil typically comprises predominantly fatty acid triglycerides, although monoglycerides and diglycerides may also be present in small amounts. The fatty acids typically have chain lengths ranging from about C12 to about C20. Representative saturated fatty acids include, for example, C12:0, C14:0, C16:0, C18:0, and C20:0 saturated fatty acids. Representative unsaturated fatty acids include, for example, C16:1, C18:1, C18:2, and C18:3 unsaturated fatty acids. Representative compositional ranges for palm oil are listed in TABLE 1.

Palm olein refers to the liquid fraction that is obtained by fractionation of palm oil after crystallization at a controlled temperature. Relative to palm oil, palm olein has a higher content of unsaturated fatty acids, for example, C18:1 and C18:2 fatty acids, and has a higher iodine value. In some embodiments, the palm olein is fractionated multiple times to produce palm olein having a higher content of unsaturated fatty acids (C18:1, C18:2) and a higher iodine value. Multiple fractionated palm olein may in some instances be referred to as palm super olein. Representative compositional ranges for palm olein are listed in TABLE 1. Representative examples of commercially available palm oil and palm oleins include those commercially available under the trade designations “SANS TRANS 25”, “SANS TRANS-39”, and “DURKEX NT100” from IOI Group, Loders Croklaan Company; and “FULLY REFINED PALM OLEIN IV 62—SUPEROLEIN” (from Cargill, Incorporated.).

Palm stearin refers to the solid fraction that is obtained by fractionation of palm oil after crystallization at controlled temperature. Relative to palm oil, palm stearin contains more saturated fatty acids and has a higher melting point. A representative composition for palm stearin is provided in TABLE 1.

TABLE 1

| | Palm Oil | Palm Olein | Palm Stearin |
|---|---|---|---|
| Fatty Acid | (% wt.) | (% wt.) | (% wt.) |
| C12:0 | <1% | <1% | <1% |
| C14:0 | <2% | <2% | <2% |
| C16:0 | 40-50% | 35-45% | 45-75% |
| C16:1 | <1% | <1% | <1% |
| C18:0 | 3-6% | 3-5% | 4-6% |
| C18:1 | 35-45% | 40-47% | 10-40% |
| C18:2 | 8-12% | 10-15% | 2-10% |
| C18:3 | <1% | <1% | <1% |
| C20:0 | <1% | <1% | <1% |
| Iodine Value (IV) | 50 to 65 | 55 to 62 | 20 to 50 |

Although the above represent typical ranges, it is understood that the amount of a given fatty acid in a palm-based oil may vary depending upon such factors as where the crop is grown, maturity of the crop, processing, weather during the growing season, etc.

### Epoxidation of Palm-Based Oils:

Oligomeric polyols of the invention are prepared from epoxidized palm-based oil compositions. Epoxidized palm-based oil compositions comprise one or more fully-epoxidized palm-based oils, one or more partially-epoxidized palm-based oils, or mixtures thereof. Optionally, the epoxidized palm-based oil composition may also include one or more epoxidized non-palm-based oils. Representative examples of epoxidized non-palm-based oils include epoxidized soybean oil, epoxidized safflower oil, epoxidized linseed oil, epoxidized corn oil, epoxidized sunflower oil, epoxidized olive oil, epoxidized canola oil, epoxidized sesame oil, epoxidized cottonseed oil, epoxidized rapeseed oil, epoxidized tung oil, epoxidized fish oil, epoxidized tallow, epoxidized peanut oil, and mixtures thereof. The non-palm-based oils may be fully epoxidized or may be partially-epoxidized. The epoxidized non-palm-based oils can be separately epoxidized and mixed with the epoxidized palm-based oil, or the non-palm-based oil can be mixed with the palm-based oil and the resulting mixture partially- or fully-epoxidized. Any useful amount of non-palm-based oil can be incorporated into the epoxidized palm-based oil composition. Representative amounts include about 10% wt. or less, about 20% wt. or less, about 30% wt. or less, about 40% wt. or less, about 50% wt. or less, about 60% wt. or less, about 70% wt. or less, about 80% wt. or less, or about 90% wt. or less.

A partially- or fully-epoxidized palm-based oil composition may be prepared by reacting a palm-based oil with a peroxyacid under conditions that convert a portion of or substantially all of the double bonds that are present in the palm-based oil to epoxide groups. Partially-epoxidized palm-based oils may include at least about 10%, at least about 20%, at least about 25%, at least about 30%, at least about 35%, at least about 40% or more of the original amount of double bonds present in the palm-based oil. The partially epoxidized palm oil may include up to about 90%, up to about 80%, up to about 75%, up to about 70%, up to about 65%, up to about 60%, or fewer of the original amount of double bonds present in the palm-based oil. Fully-epoxidized palm-based oil may include up to about 10%, up to about 5%, up to about 2%, up to about 1%, or fewer of the original amount of double bonds present in the palm-based oil.

One component of the reaction mixture is a peroxyacid. Examples of peroxyacids include peroxyformic acid, peroxyacetic acid, trifluoroperoxyacetic acid, benzyloxyperoxyformic acid, 3,5-dinitroperoxybenzoic acid, m-chloroperoxybenzoic acid, and combinations thereof. In some embodiments, peroxyformic acid or peroxyacetic acid are used. The peroxyacids may be added directly to the reaction mixture, or they may be formed in-situ by reacting a hydroperoxide with a corresponding carboxylic acid such as formic acid, benzoic acid, fatty acids (e.g., oleic acid), or acetic acid. Examples of hydroperoxides that may be used include hydrogen peroxide, tert-butylhydroperoxide, triphenylsilylhydroperooxide, cumylhydroperoxide, and combinations thereof. In an exemplary embodiment, hydrogen peroxide is used. Typically, the amount of carboxylic acid used to form the peroxyacid ranges from about 0.25 to about 1.0 moles of carboxylic acid per mole of double bonds in the palm-based oil, more typically ranging from about 0.45 to about 0.55 moles of carboxylic acid per mole of double bonds in the palm-based oil. Typically, the amount of hydroperoxide used to form the peroxy acid is about 0.5 to about 1.5 moles of hydroperoxide per mole of double bonds in the palm-based oil, more typically about 0.8 to about 1.2 moles of hydroperoxide per mole of double bonds in the palm-based oil.

In many embodiments, an additional acid component is also present in the reaction mixture. Examples of the additional acids include sulfuric acid, para-toluenesulfonic acid, trifluoroacetic acid, fluoroboric acid, Lewis acids, acidic clays, or acidic ion exchange resins.

Optionally, a solvent may be added to the reaction. Useful solvents include chemically inert solvents, for example, aprotic solvents. These solvents do not include a nucleophile and are non-reactive with acids. Hydrophobic solvents, such as aromatic and aliphatic hydrocarbons, are particularly desirable. Representative examples of suitable solvents include benzene, toluene, xylene, hexane, pentane, heptane, and chlorinated solvents (e.g., carbon tetrachloride). In an exemplary embodiment, toluene is used as the solvent. Solvents may be used to reduce the speed of reaction or to reduce the number of side reactions. In general, a solvent also acts as a viscosity reducer for the resulting composition.

After epoxidation, the reaction product may be neutralized. A neutralizing agent may be added to neutralize any remaining acidic components in the reaction product. Suitable neutralizing agents include weak bases, metal bicarbonates, or ion-exchange resins. Examples of neutralizing agents that may be used include ammonia, calcium carbonate, sodium bicarbonate, magnesium carbonate, amines, and resin, as well as aqueous solutions of neutralizing agents. Typically, the neutralizing agent will be an anionic ion-exchange resin. One example of a suitable weakly-basic ion-exchange resin is sold under the trade designation “LEWATIT MP-64” (from Bayer). If a solid neutralizing agent (e.g., ion-exchange resin) is used, the solid neutralizing agent may be removed from the epoxidized palm-based oil by filtration. Alternatively, the reaction mixture may be neutralized by passing the mixture through a neutralization bed containing a resin or other materials. Alternatively, the reaction product may be repeatedly washed to separate and remove the acidic components from the product. In addition, one or more of the processes may be combined in neutralizing the reaction product. For example, the product could be washed, neutralized with a resin material, and then filtered.

Subsequent to the epoxidation reaction, excess solvent may be removed from the reaction product. The excess solvents include products given off by the reaction, or those added to the reaction. The excess solvents may be removed by separation, vacuum, or other method. Preferably, the excess solvent removal will be accomplished by exposure to vacuum.

### Ring-Opening Oligomerization Reaction:

After preparing an epoxidized palm-based oil, the next step is to ring-open at least a portion of the epoxide groups and to oligomerize the ring-opened palm-based oil composition to form an oligomeric polyol of the invention.

Various ring-openers may be used including alcohols, water (including residual amounts of water), and other compounds having one or more nucleophilic groups. Combinations of ring-openers may also be used. Typically, the ring-opener is water, a monofunctional alcohol, or a polyfunctional alcohol (i.e., polyol). Representative examples of monofunctional alcohols include methanol, ethanol, propanol (including n-propanol and isopropanol), butanol (including n-butanol and isobutanol), and monoalkyl ethers of ethylene glycol or other glycols. Representative examples of polyfunctional alcohols include diols (e.g., ethylene glycol), triols (e.g., glycerol, trimethylolpropane), tetrols (e.g., pentaerythritol), and vegetable oil-based polyols. Any of the above monofunctional or polyfunctional alcohols may be alkoxylated.

In many embodiments, the ring-opening reaction is conducted in the presence of a ring-opening catalyst. Representative examples of ring-opening catalysts include Lewis or Brönsted acids. Examples of Brönsted acids include hydrofluoroboric acid (HBF₄), triflic acid, sulfuric acid, hydrochloric acid, phosphoric acid, phosphorous acid, hypophosphorous acid, boronic acids, sulfonic acids (e.g., para-toluene sulfonic acid, methanesulfonic acid, and trifluoromethane sulfonic acid), and carboxylic acids (e.g., formic acid and acetic acid). Examples of Lewis acids include phosphorous trichloride and boron halides (e.g., boron trifluoride). Ion exchange resins in the protic form may also be used. In an exemplary embodiment, the ring-opening catalyst is hydrofluoroboric acid (HBF₄). The ring-opening catalyst is typically present in an amount ranging from about 0.01% wt. to about 0.3% wt., more typically ranging from about 0.05% wt. to about 0.15% wt. based upon the total weight of the reaction mixture.

The ring-opening reaction is conducted under conditions that promote oligomerization of the ring-opened palm-based oil. Oligomerization may be controlled, for example, by reactant stoichiometry, degree of agitation of the reactants, and catalyst concentration. Oligomerization tends to occur to a greater degree, for example, with a high concentration of ring-opening catalyst and/or with a low concentration of ring-opener. To promote oligomerization, the ring-opener may be added in an amount that is sub-stoichiometric as compared to the number of moles of epoxide groups on the epoxidized palm-based oil. In this way, oligomerization of the epoxidized palm-based oil is favored. Specifically, during the ring-opening reaction, a portion of the ring-opened epoxide groups react with epoxide groups that are present on other molecules in epoxidized palm-based oil composition thereby resulting in oligomerization (i.e., the formation of dimers, trimers, tetramers, and higher order oligomers). The degree of oligomerization contributes to the desired properties of the oligomeric polyol including, for example, functionality, viscosity, and the distance between reactive hydroxyl groups.

In some embodiments, the stoichiometric ratio of ring-opening functional groups to epoxide groups is about 1:1 or less, for example, about 0.9:1 or less, about 0.8:1 or less, about 0.7:1 or less, about 0.6:1 or less, about 0.5:1 or less, about 0.4:1 or less, about 0.3:1 or less, about 0.2:1 or less, or about 0.1:1 or less. If the ring-opener is monofunctional (e.g., methanol), the molar amount of ring-opening functional groups is numerically equal to the molar amount of ring-opener. If the ring-opener is multifunctional (e.g., water or ethylene glycol), the molar amount of ring-opening functional groups is equal to the molar amount of ring-opener times the number of ring-opening functional groups per molecule of the ring-opener. For example, 1 mole of ethylene glycol has 2 moles of ring-opening functional groups since it is a diol. If 1 mole of ethylene glycol is used to ring-open 8 moles of epoxide groups, the stoichiometric ratio of ring-opening functional groups to epoxide groups is 2:8 or 1:4.

In some embodiments, the ring-opening reaction is stopped when the oligomeric polyol has a residual epoxy oxygen content (EOC) ranging from about 0.2% to about 2.5%, more typically ranging from about 1.0% to about 2.0%. Epoxy oxygen content refers to the weight of epoxy oxygen per molecule, expressed in percentage. For some ring-opening catalyst, the activity of the catalyst decreases over time during the ring-opening reaction. Therefore, the ring-opening catalyst may be added at a controlled rate so that the ring-opening reaction stops at, or near, the desired endpoint EOC. The ring-opening reaction may be monitored using known techniques, for example, hydroxyl number titration (ASTM E1899-02), EOC titration (AOCS Cd9-57 method) or monitoring the heat removed from the exothermic reaction.

In some embodiments, the oligomeric polyol comprises about 40% wt. or greater oligomers (including dimers, trimers, and higher order oligomers). In other embodiments, the oligomeric polyol comprises about 50% wt. or greater oligomers, for example, about 55% wt. or greater oligomers, about 60% wt. or greater oligomers, or about 65% wt. or greater oligomers. In certain embodiments, the oligomeric polyol comprises about 34.0% wt. to about 48.0% wt. monomeric polyol, about 14.0% wt. to about 18.0% wt. dimer, about 9.0% wt. to about 12.0% wt. trimer, and about 23.0% wt. or greater tetramer and higher order oligomers.

Upon completion of the ring-opening reaction, any unreacted ring-opener is typically removed, for example, by vacuum distillation. Unreacted ring-opener, especially monomers, may not be desirable in an oligomeric polyol because it may react and cap the polyisocyanate. After removing any excess ring-opener, the resulting oligomeric polyol is typically filtered to remove any solid impurities.

### Characteristics of Oligomeric Polyol:

In some embodiments, the oligomeric polyols have low hydroxyl functionality making them suitable for use in flexible slabstock polyurethane foams. Hydroxyl functionality refers to the average number of pendant hydroxyl groups (e.g., primary, secondary, or tertiary hydroxyl groups) that are present on a molecule of the oligomeric polyol. In some embodiments, the oligomeric polyols have a number average hydroxyl functionality (fₙ) of about 2.5 or less, for example about 2.4 or less, about 2.3 or less, about 2.2 or less, about 2.1 or less, about 2.0 or less, about 1.9 or less, about 1.8 or less, about 1.7 or less, about 1.6 or less, about 1.5 or less, about 1.4 or less, about 1.3 or less, about 1.2 or less, about 1.1 or less, or about 1.0 or less. Typically, the hydroxyl functionality ranges from about 1.2 to about 2.2 or from about 1.4 to about 1.9.

Another way of characterizing the number of hydroxyl groups is the hydroxyl number. Hydroxyl number refers to the number of reactive hydroxyl groups that are available for reaction with a polyisocyanate. Hydroxy number is expressed as the number of milligrams of potassium hydroxide equivalent to the hydroxyl content of one gram of the sample. Hydroxyl number can be measured, for example, using ASTM E 1899-02 standard method. In some embodiments, the oligomeric polyols of the invention have a hydroxyl number that ranges from about 40 to about 65 mg KOH/gram or from about 50 to about 55 mg KOH/gram. A hydroxyl number in the range of about 45 to about 65 mg KOH/gram is desirable because it allows the oligomeric polyol to be used in flexible slabstock polyurethane foams, where the oligomeric polyol replaces at least a portion of petroleum-derived triols that are typically used in such foams. For example, in some embodiments, the oligomeric polyol replaces at least a portion of a petroleum-derived triol having a molecular weight of about 3000 Da and a hydroxyl number of about 56 mg KOH/gram.

In some embodiments, the number average molecular weight (i.e, Mₙ) of the oligomeric polyol is about 1000 Da or greater, for example, about 1100 Da or greater, about 1200 Da or greater, about 1300 Da or greater, about 1400 Da or greater, or about 1500 Da or greater. In some embodiments, the Mₙ is less than about 5000 Da, for example, less than about 4000 Da, less than about 3000 Da, or less than about 2000 Da. In some embodiments, the Mₙ ranges from about 1000-5000 Da, for example, about 1200-3000 Da, about 1300-2000 Da, about 1700-1900 Da, or about 1500-1800 Da. Number average molecular weight may be measured, for example, using light scattering or vapor phase osmometry.

In some embodiments, the weight average molecular weight (i.e, M_{w}) of the oligomeric polyols is about 2000 Da or greater, for example, about 3000 Da or greater, about 4000 Da or greater, about 5000 Da or greater, or about 6000 Da or greater. In some embodiments, the M_{w} is less than about 20,000 Da, for example, less than about 15,000 Da, less than about 10,000 Da, or less than about 5,000 Da. In some embodiments, the M_{w} ranges from about 2000-20,000 Da, for example, about 2000-10,000 Da, or about 2500-6,000 Da. Weight average molecular weight may be measured, for example, using light scattering.

In some embodiments, the oligomeric polyols have a polydispersity (M_{w}/Mₙ) of about 1.5 to about 10.0, for example, about 1.7 to about 4.0, or about 1.9 to about 2.8.

Oligomeric polyols made from palm-based oils have a desirable balance of high oligomer content and low viscosity. In some embodiments, the oligomeric polyols have a viscosity of about 4 Pa·s or less, for example, about 3 Pa·s or less, about 2 Pa·s or less, about 1 Pa·s or less, or about 0.7 Pa·s or less. In some embodiments, the oligomeric polyols have viscosities ranging from about 0.5 Pa·s to about 2 Pa·s.

In some embodiments, the oligomeric polyols have a low acid number. Acid number refers to the amount of acidic residual material that is present in the oligomeric polyol. Acid number is reported in terms of the number of milligrams of potassium hydroxide (KOH) that is required to neutralize the acid that is present in a one gram sample of the polyol. A high acid value is undesirable because the acid may neutralize the polyurethane catalyst causing a slowing of the isocyanate-polyol reaction rate. In some embodiments, the oligomeric polyols have an acid value that is less than about 5, for example, less than about 4, less than about 3, less than about 2, or less than about 1. In exemplary embodiments, the acid value is less than about 1, for example, less than about 0.5, or from about 0.2 to about 0.5.

In some embodiments, the oligomeric polyols have few, if any, residual double bonds. This is particularly true if the oligomeric polyol is prepared from fully-epoxidized palm olein. One measure of the amount of double bonds in a substance is its iodine value (IV). The iodine value for a compound is the amount of iodine that reacts with a sample of a substance, expressed in grams iodine (I₂) per 100 grams of the substance. In some embodiments, the oligomeric polyols have an iodine value (IV) that is less than about 20 grams I₂/100 grams, for example, less than about 10 or less than about 5 grams I₂/100 grams.

### Polyurethane Compositions:

In one aspect, the invention provides polyurethane compositions that comprise the reaction product of (a) a polyisocyanate; and (b) an isocyanate-reactive composition comprising an oligomeric polyol of the invention. The hydroxyl groups present on the oligomeric polyol chemically react with the isocyanate groups of the polyisocyanate to form urethane linkages. Thus, the oligomeric polyol is chemically incorporated into a polyurethane polymer. The polyurethane compositions of the invention are useful in polyurethane foams, for example, in flexible slabstock polyurethane foams.

The amount of oligomeric polyol included in the isocyanate-reactive composition can be selected based upon the desired properties of the polyurethane. For example, in some embodiments, the isocyanate-reactive composition comprises about 10% wt. to about 90% wt. oligomeric polyol, for example, about 10% wt. to about 60% wt. oligomeric polyol, or about 15% wt. to about 40% wt. oligomeric polyol.

In some embodiments, the isocyanate-reactive composition comprises an oligomeric polyol of the invention and a petroleum-derived polyol. For example, in some embodiments, the isocyanate-reactive composition comprises about 10% wt. to about 90% wt. oligomeric polyol and about 10% wt. to about 90% wt. petroleum-derived polyol. In other embodiments, the isocyanate-reactive composition comprises about 10% wt. to about 60% wt. oligomeric polyol and about 40% wt. to about 90% wt. petroleum-derived polyol. In yet other embodiments, the isocyanate-reactive composition comprises about 15% wt. to about 40% wt. oligomeric polyol and about 60% wt. to about 85% wt. petroleum-derived polyol.

In some embodiments, the petroleum-derived polyol is a triol. As used herein, the term “triol” refers to a polyol that has an average of about 2.7 to about 3.1 hydroxyl groups per molecule. In a specific embodiment, the triol has a weight average molecular weight (Mw) of about 3000 Da to about 3500 Da. Representative examples of commercially available petroleum-derived triols include those available under the trade designations ARCOL F3040, ARCOL F3022, and ARCOL 3222 (from Bayer), PLURACOL 1385 and PLURACOL 1388 (from BASF), VORANOL 3322, VORANOL 3010, VORANOL 3136, and VORANOL 3512A (from Dow).

### Polyisocyanates:

Representative examples of useful polyisocyanates include those having an average of at least about 2.0 isocyanate groups per molecule. Both aliphatic and aromatic polyisocyanates can be used. Examples of suitable aliphatic polyisocyanates include 1,4-tetramethylene diisocyanate, 1,6-hexamethylene diisocyanate, 1,12-dodecane diisocyanate, cyclobutane-1,3-diisocyanate, cyclohexane-1,3- and 1,4-diisocyanate, 1,5-diisocyanato-3,3,5-trimethylcyclohexane, hydrogenated 2,4- and/or 4,4′-diphenylmethane diisocyanate (H₁₂MDI), isophorone diisocyanate, and the like. Examples of suitable aromatic polyisocyanates include 2,4-toluene diisocyanate (TDI), 2,6-toluene diisocyanate (TDI), and blends thereof, 1,3- and 1,4-phenylene diisocyanate, 4,4′-diphenylmethane diisocyanate (including mixtures thereof with minor quantities of the 2,4′-isomer) (MDI), 1,5-naphthylene diisocyanate, triphenylmethane-4,4′,4″-triisocyanate, polyphenylpolymethylene polyisocyanates (PMDI), and the like. Derivatives and prepolymers of the foregoing polyisocyanates, such as those containing urethane, carbodiimide, allophanate, isocyanurate, acylated urea, biuret, ester, and similar groups, may be used as well.

The amount of polyisocyanate preferably is sufficient to provide an isocyanate index of about 60 to about 120, preferably about 70 to about 110, and, in the case of high water formulations (i.e., formulations containing at least about 5 parts by weight water per 100 parts by weight of other active hydrogen-containing materials in the formulation), from about 70 to about 90. As used herein the term “isocyanate index” refers to a measure of the stoichiometric balance between the equivalents of isocyanate used to the total equivalents of water, polyols and other reactants. An index of 100 means enough isocyanate is provided to react with all compounds containing active hydrogen atoms.

### Catalyst:

Examples of useful catalysts include tertiary amine compounds and organometallic compounds. Specific examples of useful tertiary amine compounds include triethylenediamine, N-methylmorpholine, N-ethylmorpholine, diethyl ethanolamine, N-cocomorpholine, 1-methyl-4-dimethylaminoethylpiperazine, 3-methoxy-N-dimethylpropylamine, N,N-diethyl-3-diethylaminopropylamine, dimethylbenzylamine, bis(2-dimethylaminoethyl)ether, and the like. Tertiary amine catalysts are advantageously used in an amount from about 0.01 to about 5, preferably from about 0.05 to about 2 parts per 100 parts by weight of the active hydrogen-containing materials in the formulation.

Specific examples of useful organometallic catalysts include organic salts of metals such as tin, bismuth, iron, zinc, and the like, with the organotin catalysts being preferred. Suitable organotin catalysts include dimethyltindilaurate, dibutyltindilaurate, stannous octoate, and the like. Other suitable catalysts are taught, for example, in U.S. Pat. No. 2,846,408, which is hereby incorporated by reference. Preferably, about 0.001 to about 1.0 parts by weight of an organometallic catalyst is used per 100 parts by weight of the active hydrogen-containing materials in the formulation. Blends of catalysts may also be used.

### Blowing Agent:

The blowing agent generates a gas under the conditions of the reaction between the active hydrogen compound and the polyisocyanate. Suitable blowing agents include water, liquid carbon dioxide, acetone, and pentane, with water being preferred.

The blowing agent is used in an amount sufficient to provide the desired foam density and IFD. For example, when water is used as the only blowing agent, from about 0.5 to about 10, preferably from about 1 to about 8, more preferably from about 2 to about 6 parts by weight, are used per 100 parts by weight of other active hydrogen-containing materials in the formulation.

### Other Additives:

Other additives that may be included in the formulation include surfactants, catalysts, cell size control agents, cell opening agents, colorants, antioxidants, preservatives, static dissipative agents, plasticizers, crosslinking agents, flame retardants, and the like.

Examples of useful surfactants include silicone surfactants and the alkali metal salts of fatty acids. The silicone surfactants, e.g., block copolymers of an alkylene oxide and a dimethylsiloxane, are preferred, with “low fog” grades of silicone surfactants being particularly preferred.

In some cases, a static dissipative agent may be included in the formulation during foam preparation, or used to treat the finished foam. Useful examples include non-volatile, ionizable metal salts, optionally in conjunction with an enhancer compound, as described in U.S. Pat. Nos. 4,806,571, 4,618,630, and 4,617,325. Of particular interest is the use of up to about 3 weight percent of sodium tetraphenylboron or a sodium salt of a perfluorinated aliphatic carboxylic acid having up to about 8 carbon atoms.

### Foam Manufacturing:

In some embodiments, the polyurethane compositions are suitable for flexible slabstock polyurethane foams. Flexible slabstock polyurethane foams can be manufactured using conventional slabstock foaming equipment, for example, commercial box-foamers, high or low pressure continuous foam machines, crowned block process, rectangular block process (e.g., Draka, Petzetakis, Hennecke, Planiblock, EconoFoam, and Maxfoam processes), or verti-foam process. In some embodiments, the slabstock foam is produced under reduced pressure. For example, in variable pressure foaming (VPF), the complete conveyor section of the foaming machine is provided in an airtight enclosure. This technique allows for the control of foam density and the production of foam grades that may otherwise be difficult to produce. Details of flexible slabstock polyurethane foams and slabstock foaming processes are reported, for example, in Chapters 5 and 9 of Flexible Polyurethane Foams, edited by Herrington and Hock, (2^{nd} Edition, 1997, Dow Chemical Company).

The invention has been described with reference to various specific and preferred embodiments and techniques. It will be understood, however, that reasonable modifications of such embodiments and techniques can be made while remaining within the spirit and scope of the invention.

### EXAMPLES

### Example 1

This Example describes the preparation and characterization of oligomeric polyols of the invention.

### Characterization of Palm-Based Oil Samples:

TABLE 2 lists measured iodine value (IV), viscosity, and fatty acid composition (SFA=saturated fatty acids; MUFA=monounsaturated fatty acids; and PUFA=polyunsaturated fatty acids) for three palm-based oils.

TABLE 2

| | | Viscosity | | | | |
|---|---|---|---|---|---|---|
| | | at 25° C. | Iodine | SFA | MUFA | PUFA |
| Sample | Type | (Pa · s) | Value | (%) | (%) | (%) |
| SANSTRANS25 | Palm | 0.09 | 55.43 | 44.9 | 44.5 | 10.0 |
| | Olein | | | | | |
| SANSTRANS39 | Palm | — | 51.56 | 50.7 | 39.6 | 9.6 |
| | Oil | | | | | |
| DURKEX NT100 | Palm | 0.09 | 60.68 | 42.1 | 44.6 | 13.1 |
| | Super | | | | | |
| | Olein | | | | | |

### Step 1: Epoxidation of Palm-Based Oil

300 grams of DURKEX NT100 was epoxidized by peroxoacetic acid formed in situ. Epoxidation was carried out at 70° C. using a water bath. The molar ratio of (double bonds in the palm olein):(H₂O₂):acetic acid was 1:1.5:0.5. The catalyst used was the ion exchange resin (AMBERLITE IR 120) added in amount of 25% by wt. of the water phase. Toluene was used as the solvent for the oil. The total epoxidation time was 9 hours. Samples of the epoxidized reaction product were taken at 1; 3, 5 and 7 hours. The samples were purified by washing with warm water and were analyzed for epoxy oxygen content (EOC), iodine value (IV), viscosity, and MWD. TABLE 3 details the result of the analyses.

TABLE 3

| | | | | Viscosity at |
|---|---|---|---|---|
| Time (Hr) | EOC (% O) | Iodine Value | Selectivity | 25° C. (Pa · s) |
| 0 | 0 | 60.7 | | 0.09 |
| 1 | 1.76 | 32.5 | 1.03 | 0.11 |
| 3 | 3.12 | 9.7 | 1.01 | 0.15 |
| 5 | 3.46 | 3.6 | 1.00 | 0.16 |
| 7 | 3.60 | 1.2 | 1.00 | 0.17 |
| 9 | 3.60 | 0.6 | 0.99 | 0.17 |

From TABLE 3 it can be seen that the epoxidation went almost quantitatively. The EOC reached a plateau of 3.60% O, which correlates to an epoxy yield of 98%. Selectivity of about 1.0 throughout the reaction indicated that no side-reactions were occurring. The absence of side-reactions was confirmed by GPC.

### Step 2: Ring-Opening Oligomerization

Three oligomeric polyols of the invention were prepared from the fully-epoxidized palm olein that was prepared as described in Step 1 of this Example. Methanol or water was used as the ring-opener. Hydrofluoroboric acid (HBF₄) was used as the ring-opening catalyst. The amount of the reactants used and the molar ratio of the ring-opener to epoxide are shown in TABLE 4.

TABLE 4

| | Epoxidized | | | HBF₄ | |
|---|---|---|---|---|---|
| | Palm Olein | | | (g of 48% | Ring- |
| | (g) | Methanol | Water | wt. | opener to |
| | [mole | (g) | (g) | solution) | epoxide |
| Sample | epoxide] | [mol] | [mol] | [%] | molar ratio |
| 1-A | 70.0 | 2.02 | | 0.15 | 0.4 |
| | [0.158] | [0.063] | | [0.1] | |
| 1-B | 70.0 | 2.02 | | 0.075 | 0.4 |
| | [0.158] | [0.063] | | [0.05] | |
| 1-C | 70.0 | | 0.57 | 0.15 | 0.2 |
| | [0.158] | | [0.0315] | [0.1] | |

The reactants were mixed in a three-necked round-bottom flask at room temperature and the exotherm was monitored. After mixing, the reaction mixture was heated in a water bath to 70° C. and the reaction was allowed to proceed for about 1 hour. In the case of water as the ring-opener (see, 1-C), the exothermic effect occurred during the heating stage when the temperature of the reaction mixture reached about 35° C. At the end of the reaction, the mixture was diluted with acetone (no neutralization of catalyst) and was filtered. Solvent and unreacted ring-opener were removed using a rotary evaporator. The resulting polyols were low viscosity, bright yellow-colored liquids at room temperature. The polyols were characterized and the results are reported in TABLE 5.

TABLE 5

| | OH | OH Number | | | | | |
|---|---|---|---|---|---|---|---|
| | Number | Max* | | | | | |
| | (mg | (mg | EOC | Acid | Viscosity | Oligomers | Conversion** |
| Sample | KOH/gram) | KOH/gram) | (%) | Number | (Pa · s) | (%) | (%) |
| 1-A | 47.27 | 54.6 | 1.04 | 0.78 | 1.62 | 65.1 | 87% |
| 1-B | 41.22 | 51.6 | 1.69 | 2.28 | 0.66 | 51.9 | — |
| 1-C | 51.23 | 56.8 | 1.91 | 0.23 | 0.94 | 54.3 | 90% |

*OH Number Max was calculated taking into account water from the catalyst solution.
**Conversion (%) = (OH Number/OH Number Max) × 100

It was observed that a higher conversion was obtained when 0.1% HBF₄ catalyst was used. The palm olein based oligomeric polyols had a high content of oligomers yet displayed a very low viscosity.

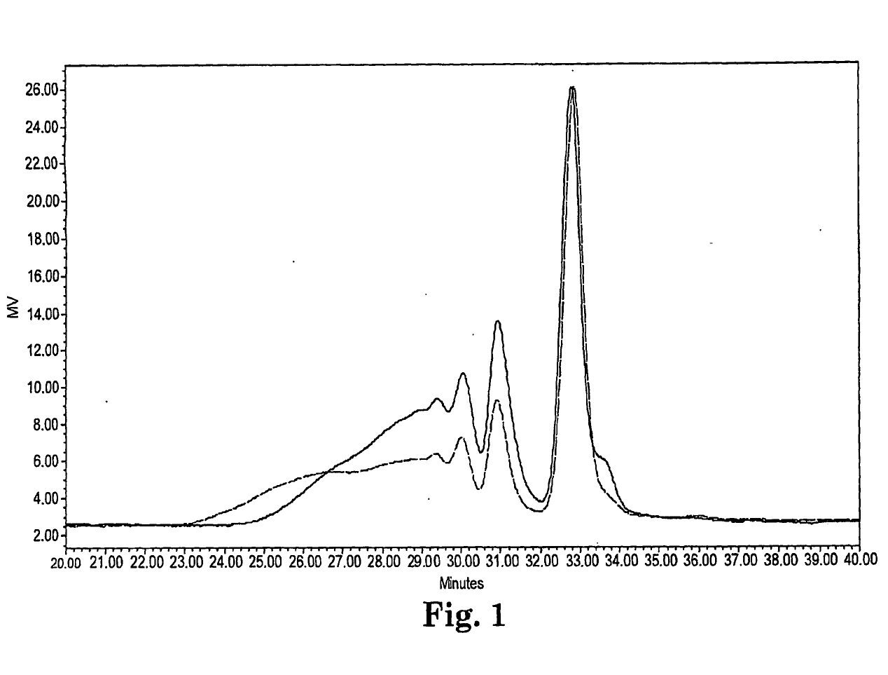

FIG. 1 is a GPC chromatogram trace of polyol 1-A (—) as compared to a polyol prepared using fully epoxidized RBD soybean oil ring opened with methanol at a sub-stoichiometric ratio (see, WO 2006/012344A1) (— —). Although not wishing to be bound by theory, it is believed that the low viscosity of palm-polyols is due to the absence of very high MW oligomers and the presence of a significant fraction of low MW oligomers.

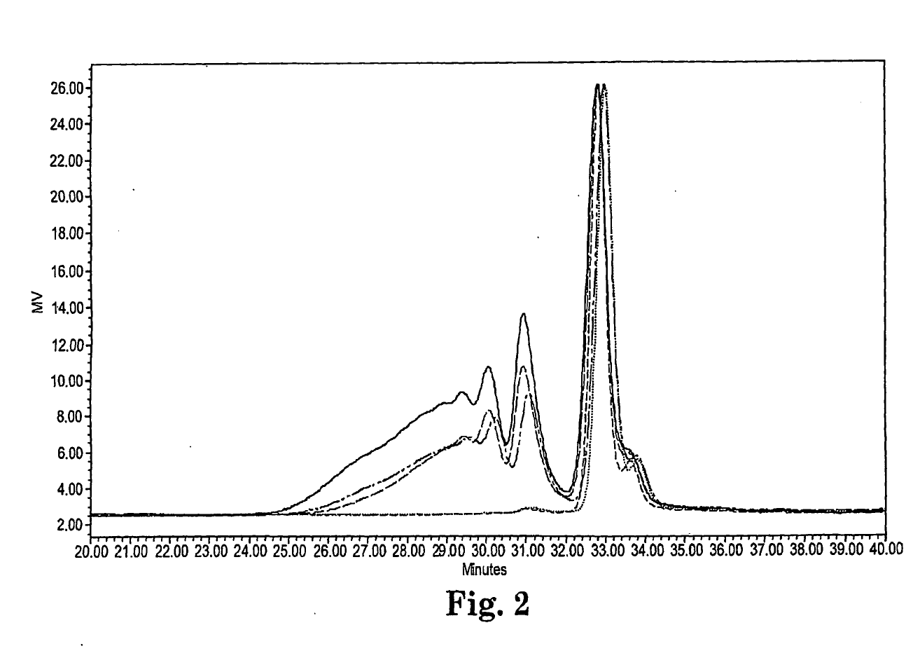

FIG. 2 is a GPC chromatogram trace of oligomeric polyols of the invention 1-A (—) 1-B (— —), and 1-C (— • • —). Also shown are GPC chromatogram traces of DURKEX NT100 palm olein (. . .) and epoxidized DURKEX NT100 palm olein (- - -).

### Example 2

Foams comprising the oligomeric polyols of Example 1 were prepared and tested as described in this Example 2.

A 400 ml plastic beaker was positioned on an electric scale. Next, the formulation required amount of polyol(s) were added to the beaker. Next, the formulation required amount of silicone surfactant and amine catalyst were added to the beaker. Next, the formulation required amount of tin catalyst and water were added to the batch. The temperature of the B-side was adjusted so that upon mixing with the polyisocyanate the combined mixture had a temperature of 19.2°±0.3° C. The batch was mixed with an electric, lab duty mixer (Delta ShopMaster brand, Model DP-200, 10 inch shop drill press) equipped with a 2″ diameter mixing blade (ConnBlade Brand, Model ITC from Conn Mixers Co.) for 19 seconds at 2340 rpm.

Separately, the formulation required amount of TDI was weighed out into a 50 ml plastic beaker and was set near the mixing station. The TDI was then added to the polyol mixture and was mixed for 6 seconds. Following this, the mixture was poured into an 83 ounce cup and was allowed to free rise. During the free rise period, the Cream Time (i.e., the time from the introduction of the TDI until start of cream rise in the cup), Top of Cup Rise Time (i.e., the time from the introduction of the TDI until the dome of the foam reaches the top of the cup), and the Total Rise Time (i.e., the time from the introduction of the TDI until there is blow-off or no more rising of the foam) were each recorded. The foam and cup were then placed into a temperature-controlled oven at 100° C. for 15 minutes to cure. At the end of the oven cure, the foam was permitted to cure overnight. After curing overnight, the foam was conditioned for 72 hours at 25° C. and 50% relative humidity before testing for physical properties. The physical property test results are reported in TABLE 6.

### Ingredient List

Polyol F3022—a petroleum-derived, nominal 3000 molecular weight triol having a hydroxyl number of 54.3 mg KOH/g and an acid number of 0.03 mg KOH/gram (commercially available under the trade designation “ARCOL F-3022” from Bayer).
Amine BL11—a blowing catalyst consisting of 70% bis(dimethylaminoethyl)ether and 30% dipropylene glycol (commercially available under the trade designation “DABCO BL-11” from Air Products).
Tin K29—stannous octoate catalyst (commercially available from Degussa).
Silicone EP-H-140—silicone surfactant.
TDI—toluene diisocyanate.

TABLE 6

| | Control | 2-A1 | 2-A2 | 2-B1 | 2-B2 | 2-C1 | 2-C2 |
|---|---|---|---|---|---|---|---|
| Polyurethane | | | | | | | |
| Formulations | | | | | | | |
| B-Side | | | | | | | |
| Polyol F3022 | 100 | 80 | 70 | 80 | 70 | 70 | 74 |
| Polyol 1-A | 0 | 20 | 30 | 0 | 0 | 0 | 0 |
| Polyol 1-B | 0 | 0 | 0 | 20 | 30 | 0 | 0 |
| Polyol 1-C | 0 | 0 | 0 | 0 | 0 | 30 | 26 |
| H₂O (pph) | 3.98 | 3.98 | 3.98 | 3.98 | 3.98 | 3.98 | 3.98 |
| Silicone | 1.0 | 1.0 | 1.0 | 1.0 | 1.0 | 1.0 | 1.0 |
| EP-H-140 | | | | | | | |
| (pph) | | | | | | | |
| Tin K29 | 0.22 | 0.22 | 0.22 | 0.22 | 0.22 | 0.22 | 0.22 |
| (pph) | | | | | | | |
| Amine BL11 | 0.16 | 0.16 | 0.16 | 0.16 | 0.16 | 0.16 | 0.16 |
| (pph) | | | | | | | |
| A-Side | | | | | | | |
| TDI | 105 | 105 | 105 | 105 | 105 | 105 | 105 |
| (index) | | | | | | | |
| TDI | 49.6 | 49.2 | 48.9 | 49.4 | 49.2 | 49.4 | 49.4 |
| (pph) | | | | | | | |
| Properties of | | | | | | | |
| Polyurethane | | | | | | | |
| Composition | | | | | | | |
| Cream time (sec) | 10.97 | 13 | 16 | 13 | 14 | 14 | 14 |
| Top of Cup | 40.27 | 51 | 59 | 49 | 55 | 53 | 55 |
| time | | | | | | | |
| (sec) | | | | | | | |
| Rise time | 78.6 | 104 | 117 | 98 | 105 | 107 | 105 |
| (sec) | | | | | | | |
| Tack-free | | Good | Good | Good | Good | Good | Good |
| Time | | | | | | | |
| Foam Odor | | Bad | Bad | Bad | Bad | Bad | Bad |
| Physical | | | | | | | |
| Properties of | | | | | | | |
| Polyurethane | | | | | | | |
| Foam | | | | | | | |
| Density | 1.50 | 1.55 | — | 1.51 | 1.62 | 1.56 | 1.52 |
| (lb/ft³) | | | | | | | |
| Resiliency | 42.8 | 41 | — | 40.3 | 36.7 | 37.3 | 39.3 |
| (%) | | | | | | | |
| 25% IFD | 26.55 | 24.31 | — | 25.61 | 26.25 | 20.79 | 22.94 |
| (N) | | | | | | | |
| 65% IFD | 45.99 | 44.88 | — | 50.23 | 50.31 | 45.42 | 44.31 |
| (N) | | | | | | | |
| Support Factor | 1.73 | 1.85 | — | 1.96 | 1.92 | 2.18 | 1.93 |
| IFD | 33.03 | 30.25 | — | 31.87 | 32.66 | 25.87 | 28.55 |
| (15″ by 15″ by | | | | | | | |
| 4″) | | | | | | | |
| (lbs/50 in²) | | | | | | | |
| Tensile | 121 | 96.59 | 59.87 | 93.81 | 86.93 | 92.47 | 87.54 |
| (kPa) | | | | | | | |
| Elongation | 188 | 150.06 | 99.04 | 170.88 | 131.82 | 107.21 | 114.0 |
| (%) | | | | | | | |
| Tear | 516 | 365.00 | — | 365.00 | 290.00 | 210.00 | 270.00 |
| (N/m) | | | | | | | |
| Permeability | 3.41 | 3.58 | — | 3.83 | 2.94 | 2.83 | 3.25 |
| (ft³/min) | | | | | | | |
| 90% CS (% loss) | 10.99 | 8.02 | — | 7.54 | 8.11 | 7.58 | 7.12 |

### Example 3

Oligomeric polyols of the invention were prepared from a palm-based oil composition comprising a 1:1 wt./wt. mixture of epoxidized palm olein and epoxidized soybean oil. The composition had an EOC of 5.23%. The palm-based oil composition was ring-opened with either methanol (3-A) or water (3-B). Hydrofluoroboric acid was used as the ring-opening catalyst. The amount of the reactants used and the mole ratio of the ring-opener to epoxide are shown in TABLE 7.

TABLE 7

| | Mixture of | | | | |
|---|---|---|---|---|---|
| | Epoxidized | | | | |
| | Palm Olein and | | | HBF₄ | |
| | Epoxidized | | | (g of | Ring- |
| | Soybean Oil | Methanol | Water | 48% wt. | opener to |
| | (g) | (g) | (g) | solution) | epoxide |
| Sample | [mole epoxide] | [mol] | [mol] | [%] | mole ratio |
| 3-A | 80.0 | 3.35 | | 0.087 | 0.4 |
| | [0.262] | [0.105] | | [0.05] | |
| 3-B | 80.0 | | 0.94 | 0.17 | 0.2 |
| | [0.262] | | [0.052] | [0.1] | |

The polyols were cloudy liquids at room temperature. The polyols were characterized and the results are reported in TABLE 8.

TABLE 8

| | | OH Number | | | |
|---|---|---|---|---|---|
| | OH Number | Max* | | | |
| | (mg | (mg | | Viscosity | Oligomers |
| Sample | KOH/gram) | KOH/gram) | EOC (%) | (Pa · s) | (%) |
| 3-A | 60.3 | 79.9 | 2.59 | 1.30 | 51.6 |
| 3-B | 63.3 | 86.2 | 2.87 | 2.50 | 55.6 |

*OH Number Max was calculated taking into account water from the catalyst solution.

GPC analysis of the oligomeric polyols yielded the distribution of oligomers shown in TABLE 9.

TABLE 9

| | | | | Tetramers | |
|---|---|---|---|---|---|
| | | | | and Higher | |
| | | | | Order | Total |
| Sample | Monomers | Dimers | Trimers | Oligomers | Oligomers |
| 3-A | 48.4 | 14.9 | 8.8 | 28.7 | 51.6 |
| 3-B | 44.4 | 10.6 | 7.1 | 37.9 | 55.6 |

### Example 4

Example 4 describes the preparation and characterization of an oligomeric palm-based polyol of the invention.

### Step 1: Epoxidation:

To a 22-Liter 5-neck round-bottom flask equipped with a thermocouple, heating mantle, temperature controller, an internal Teflon-coated cooling coil, and a nitrogen sweep was charged 6,001 grams of DURKEX NT 100 (palm super olein, 63 IV, 14.9 moles C═C), 447 g glacial acetic acid (7.45 moles), 745 grams of AMBERLITE IR 120H, and 3,000 grams of toluene. The reaction mixture has heated with stirring to 70° C. The heat was turned off and a solution of 30% aqueous hydrogen peroxide was added at ˜20 grams/minute. A total of 2,531 g of 30% peroxide (22.33 moles) was added over two hours. Cooling water flow through the cooling coil was adjusted to maintain a temperature of 70° C.±2° C. To maintain 70° C., cooling was required for the first 4.5 hours of reaction, after which heating was required. The reaction was monitored by measuring the epoxide oxygen content (% EOC) of the toluene diluted product. The % EOC versus time is provided in TABLE 10. The stirring and cooling were stopped after 10 hours.

TABLE 10

| | % Epoxide Oxygen |
|---|---|
| Hours | (% EOC) |
| 0 | 0 |
| 1 | 0.76 |
| 2 | 1.47 |
| 3 | 1.89 |
| 4 | 2.19 |
| 5 | 2.3 |
| 6 | 2.39 |
| 7 | 2.41 |
| 8 | 2.43 |
| 9 | 2.45 |

Next, the aqueous and organic phases were allowed to separate. The AMBERLITE IR 120H settled to the bottom with the aqueous phase. The aqueous phase and the AMBERLITE resin were sucked out of the flask, and the organic phase was washed successively with ˜3,000 grams of 60° C. water until the water phase had a pH of 7 (5 washes).

The washed product was stripped under vacuum to final conditions of <5 Torr at 90° C. A total of 6,036 grams of epoxidized palm super olein (ESPO) were obtained (97% yield, not allowing for sampling and transfer losses.) The ESPO had a % EOC of 3.61% and an acid value of 0.36 mg KOH/gram. The ESPO partially solidified upon standing at room temperature.

### Step 2: Epoxide Ring Opening by Methanol

An oligomeric polyol was prepared from the EPSO described above in a 12-Liter, 5-neck round-bottom flask equipped with a two-level agitator, thermocouple, heating mantle, cooling coil, a water-cooled condenser, and a nitrogen sweep. The flask was charged with 4,000 grams of ESPO (9.03 moles epoxide) and 88.4 g (2.76 moles) of methanol and was heated to 55° C. with stirring. A catalyst solution of 28.1% of 48% HBF₄ in methanol (13.5% HBF₄) was added subsurface to the reaction mixture through a stainless steel (316SS) tube at a rate of ˜0.14 mL/minute (0.13 g/min). Heating was required to maintain 55° C. for the first approximately 1.5 hours, but cooling was required thereafter. The % EOC of the reaction mixture was measured at 1-hour intervals. Catalyst addition was stopped when the EOC reached 1.13%. The % EOC versus time is shown in TABLE 11. The total HBF₄ over 5 hours was 1265 ppm relative to EPSO and methanol. The total methanol charge including catalyst was 116 g (3.63 moles), corresponding to a MeoH/epoxide mole ratio of 0.40.

TABLE 11

| Hours | % EOC |
|---|---|
| 0 | 3.51 |
| 0.5 | 3.47 |
| 1.0 | 3.22 |
| 1.5 | 2.94 |
| 2.0 | 2.77 |
| 2.5 | 2.53 |
| 3.0 | 2.22 |
| 3.5 | 2.04 |
| 4.0 | 1.73 |
| 4.5 | 1.48 |
| 5.0 | 1.27 |
| 5.5 | 1.13 |
| 6.0 | 1.08 |

The partially ring-opened product was stripped to final conditions of <5 Torr at 88° C. The resulting clear pale yellow liquid product had the properties listed in TABLE 12.

TABLE 12

| Property | Value | |
|---|---|---|
| Gardner Color | 3 | |
| Hydroxyl Number | 39 | mg KOH/gram |
| Number Ave. Hydoxyl | 1.34 | |
| Functionality (VPO) | | |
| Epoxide Oxygen content | 1.01% | |
| Acid Value | 0.91 | mg KOH/gram |
| Viscosity | 2.371 Pa · s @ 25° C. | |
| Water | 108 | ppm |
| Oligomer Content | 70.7% | (GPC) |

### Example 5

### Step 1

### Epoxidation of Palm Olein

300 grams of palm olein was epoxidized by peroxoacetic acid formed in situ. Epoxidation was carried out at 70° C. (water bath). Molar ratio of double bonds:H₂O₂:acetic acid was 1:1.5:0.5. The catalyst used was the ion exchange resin AMBERLITE IR 120, added in an amount of 25% by weight of the water phase. Toluene was used as the solvent for the oil. The total time of epoxidation was 9 hours. Samples of the reaction mixture were taken at 1, 3, 5 and 7 hours, purified by multiple washings with warm water, and analyzed for EOC, I.V., and viscosity. TABLE 13 lists the results of the analyses.

TABLE 13

| Time | EOC | | | Viscosity at |
|---|---|---|---|---|
| (Hr) | (% O) | Iodine value | Selectivity | 25° C., (Pa · s) |
| 0 | 0 | 55.43 | | 0.09 |
| 1 | 1.52 | 39.23 | 1.54 | 0.12 |
| 3 | 2.98 | 3 | 0.93 | 0.14 |
| 5 | 3.31 | −0.56 | 0.97 | 0.15 |
| 7 | 3.37 | 0.14 | 1.00 | 0.16 |
| 9 | 3.39 | −0.97 | 0.99 | 0.16 |

From TABLE 13 it can be seen that epoxidation went quantitatively. The % EOC reached a plateau of 3.39% O, which is 100% of epoxy yield, indicating that no side reactions took place. The absence of side reactions during epoxidation was confirmed by GPC.

### Step 2

### Ring Opening Oligomerization

The polyol was prepared from the above fully epoxidized palm olein. Methanol was used as a ring-opener and tetrafluoroboric acid (HBF₄) as the catalyst. Composition of the reaction mixture is given in Table 14. Reactants were mixed in a 250 mL three-necked RB flask at room temperature and the exothermic effect was monitored. After that, the reaction mixture was heated (water bath) to 70° C. and allowed to proceed for 1 hour.

TABLE 14

| | Epoxidized | | | Ring-opener |
|---|---|---|---|---|
| | palm olein | Methanol | HBF₄ g of 48% | to epoxy |
| Sample ID | g (mol epoxy) | g (mol) | solution (%) | mole ratio |
| 5 | 70 (0.148) | 1.90 (0.059) | 0.15 (0.1) | 0.4 |

At the end of the reaction, the mixture was diluted with acetone (no neutralization of catalyst) and filtered. The solvent and unreacted ring-opener were removed on a rotary evaporator. The polyol was characterized and the results are reported in TABLES 15 & 16.

TABLE 15

| | | OH# | | | Acid | |
|---|---|---|---|---|---|---|
| | OH# | Max* | | | value | |
| | (mg | (mg | EOC | Viscosity | (mg | Oligomers |
| Sample ID | KOH/g) | KOH/g) | (% O) | (Pa s) | KOH/g) | (%) |
| 5 | 43.5 | 56.5 | 0.93 | 1.15 | 0.46 | 62.6 |

OH#ₘₐₓ was calculated taking into account water from the catalyst solution.

TABLE 16

| Sample | OH# | | | | |
|---|---|---|---|---|---|
| ID | (mgKOH/g) | Mn | fₙ | Mw | Mw/Mn |
| 5 | 43.5 | 1,944 | 1.51 | 4,483 | 2.3 |

### Example 6

### Step 1

### Epoxidation of RBD Palm Oil (SANS TRANS 39)

300 grams of palm oil was epoxidized by peroxoacetic acid formed in situ. Epoxidation was carried out at 70° C. (water bath). The molar ratio of double bonds:H₂O₂:acetic acid was 1:1.5:0.5. The catalyst used was the ion exchange resin AMBERLITE IR 120, added in an amount of 25% by weight of the water phase. Toluene was used as the solvent for the oil. The reaction mixture composition is shown in TABLE 17. The total time of epoxidation was 9 hours. Samples of the reaction mixture were taken at 1, 3, 5 and 7 hours, purified by multiple washings with warm water, and analyzed for EOC, I.V., and viscosity. TABLE 17 lists the results of the analyses.

TABLE 17

| | | | | | Viscosity at |
|---|---|---|---|---|---|
| Time | EOC | | | OH# | 25° C. |
| (Hr) | (% O) | Iodine value | Selectivity | (mg KOH/g) | (Pa · s) |
| 0 | 0 | 51.56 | | 7.67 | 0.09 |
| 1 | 1.61 | 26.64 | 1.06 | | 0.11 |
| 3 | 2.65 | 9.09 | 1.02 | | 0.15 |
| 5 | 3.00 | 3.45 | 1.02 | | 0.16 |
| 7 | 3.12 | 0.02 | 0.99 | | 0.17 |
| 9 | 3.17 | 0.10 | 1.01 | 7.82 | 0.17 |

As can be seen, the epoxidation of palm oil was quantitative. The % EOC reached the maximum value of 3.17% O, and the selectivity was around 1 throughout the whole process of epoxidation, indicating no side reactions.

### Step 2

### Ring Opening Oligomerization

Fully epoxidized palm oil was used for preparation of the polyols 6-A and 6-B. TABLE 18 shows the amounts of reagents used.

TABLE 18

| | Epoxidized | | | Ring-opener |
|---|---|---|---|---|
| | palm oil | Methanol | HBF₄ g of 48% | to epoxy |
| Sample ID | g (mol epoxy) | g (mol) | solution (%) | mole ratio |
| 6-A | 70 (0.139) | 2.22 (0.069) | 0.15 (0.1) | 0.5 |
| 6-B | 70 (0.139) | 2.22 (0.069) | 0.30 (0.2) | 0.5 |

The reactants were mixed in a three-necked RB flask at room temperature and the exotherm was monitored. The reaction mixture was then heated (water bath) to 70° C. and allowed to proceed for 1 hour. At the end of the reaction, the mixture was diluted with acetone (no neutralization of catalyst) and filtered. The solvent and unreacted ring-opener were removed on a rotary evaporator. The polyols were analyzed and these results are shown in TABLES 19 and 20.

TABLE 19

| | | | | | Acid | |
|---|---|---|---|---|---|---|
| | OH# | OH#max* | | | value | |
| Sample | (mg | (mg | EOC | Viscosity | (mg | Oligomers |
| ID | KOH/g) | KOH/g) | (% O) | (Pa · s) | KOH/g) | (%) |
| 6-A | 46.3 | 66.0 | 1.00 | 0.74 | 0.90 | 50.76 |
| 6-B | 61.8 | 71.8 | 0.23 | 1.80 | 0.68 | 59.68 |

*OH#ₘₐₓ was calculated taking into account water from the catalyst solution.

TABLE 20

| | OH# | | | | | Oligomers |
|---|---|---|---|---|---|---|
| Sample ID | (mgKOH/g) | Mn | fₙ | Mw | Mw/Mn | (%) |
| 6-A | 46.3 | 1285 | 1.06 | 2497 | 1.9 | 50.8 |
| 6-B | 61.8 | 1976 | 2.18 | 4970 | 2.8 | 59.7 |

### Example 7

### Step 1

### Epoxidation of RBD Palm Olein (SANS TRANS 25)

Epoxidation of palm olein by peroxoacetic acid formed in situ went quantitatively with good selectivity as in the case of palm oil and palm super olein. The compositions of the reaction mixtures are given in TABLE 21, and properties of the epoxidized palm olein are given in TABLE 22.

TABLE 21

| | SANS TRANS | Acetic | H₂O₂ | AMBERLITE | |
|---|---|---|---|---|---|
| | 25 | Acid | solution | IR 120 | Toluene |
| Sample ID | (g) | (g) | (g) | (g) | (g) |
| EPOlein | 600 | 39.3 | 223 | 65.5 | 300 |

TABLE 22

| | Time | EOC | Iodine | | Viscosity at 25° C. |
|---|---|---|---|---|---|
| | (Hr) | (% O) | value | Selectivity | (Pa · s) |
| SansTrans25 | 0 | 0 | 55.43 | | 0.09 |
| Epox Palm | 9 | 3.36 | 0.4 | 1.00 | 0.16 |
| Olein | | | | | |

### Step 2

### Ring Opening Oligomerization

Five polyols were synthesized from epoxidized palm olein using methanol, water and monomethyl ether of ethylene glycol (methyl cellosolve) as ring-openers. TABLE 23 lists the composition of the reaction mixtures.

TABLE 23

| | Epoxidized | | | Methyl | HBF₄ | |
|---|---|---|---|---|---|---|
| | Palm olein | Methanol | Water | Cellosolve | g of 48% | Ring-opener |
| | g | g | g | g | solution | to epoxy |
| Sample ID | (mol epoxy) | (mol) | (mol) | (mol) | (%) | molar ratio |
| 7-A | 70 | | 0.53 | | 0.15 | 0.2 |
| | (0.148) | | (0.030) | | (0.1) | |
| 7-B | 70 | | 0.80 | | 0.15 | 0.3 |
| | (0.148) | | (0.044) | | (0.1) | |
| 7-C | 300 | 8.06 | | | 0.64 | 0.4 |
| | (0.630) | (0.252) | | | (0.1) | |
| 7-D* | 300 | | 3.55 | | 0.63 | 0.2 |
| | (0.986) | | (0.197) | | (0.1) | |
| 7-E | 150 | | | 11.97 | 0.34 | 0.5 |
| | (0.315) | | | (0.158) | (0.1) | |

*Sample 7-D was a 50/50 wt. mixture of epoxidized soybean oil and epoxidized palm olein (EOC of mixture = 5.26% O).

The polyols were characterized and the results are reported in TABLES 24 and 25.

TABLE 24

| | | OH# | | | | |
|---|---|---|---|---|---|---|
| | OH# | max | | | Acid value | |
| Sample | (mg | (mg | EOC | Viscosity | (mg | Oligomers |
| ID | KOH/g) | KOH/g) | (% O) | (Pa · s) | KOH/g) | (%) |
| 7-A | 49.0 | 59.1 | 1.70 | 0.84 | 0.53 | 52.5 |
| 7-B | 69.49 | 82.4 | 1.38 | 1.35 | 0.72 | 56.5 |
| 7-C | 47.8 | 56.1 | 1.09 | 0.97 | 0.93 | 58.7 |
| 7-D | 69.9 | 84.8 | 2.89 | 3.12 | 1.72 | 58.2 |
| 7-E | 53.41 | 64.7 | 1.11 | 0.73 | 1.17 | 54.4 |

TABLE 25

| | OH# | | | | |
|---|---|---|---|---|---|
| Sample ID | (mgKOH/g) | Mn | fₙ | Mw | Mw/Mn |
| 7-A | 49.04 | 1505 | 1.32 | 3572 | 2.4 |
| 7-B | 69.49 | 1495 | 1.85 | 3552 | 2.4 |
| 7-C | 47.8 | 1739 | 1.48 | 3667 | 2.1 |
| 7-D | 69.9 | 1773 | 2.21 | 10330 | 5.8 |
| 7-E | 53.41 | 1677 | 1.60 | 2981 | 1.8 |

All publications and patents mentioned herein are hereby incorporated by reference. The publications and patents disclosed herein are provided solely for their disclosure. Nothing herein is to be construed as an admission that the inventors are not entitled to antedate any publication and/or patent, including any publication and/or patent cited herein.

Other embodiments of this invention will be apparent to those skilled in the art upon consideration of this specification or from practice of the invention disclosed herein. Various omissions, modifications, and changes to the principles and embodiments described herein may be made by one skilled in the art without departing from the true scope and spirit of the invention which is indicated by the following embodiments.

1-56. (canceled)

57. An oligomeric polyol comprising:
a ring-opened and oligomerized epoxidized palm-based oil composition that comprises about 40% wt. or greater oligomers, a hydroxyl number of from about 40 to about 65, a number average hydroxyl functionality of about 2.5 or less, and a viscosity at 25° C. of about 2 Pa·s or less.

58. The oligomeric polyol of claim 57, wherein the palm-based oil composition is made from an oil comprising palm olein.

59. The oligomeric polyol of claim 58, wherein the palm-based oil composition is made from an oil comprising palm super olein.

60. The oligomeric polyol of claim 1, wherein the ring-opened and oligomerized palm-based oil composition is made from a fully-epoxidized palm-based oil composition.

61. The ring-opened and oligomerized palm-based oil composition of claim 57, wherein the composition has a viscosity at 25° C. of about 1 Pa·s or less.

62. The ring-opened and oligomerized epoxidized palm-based oil composition of claim 57, wherein the composition has about 50% wt. oligomers or greater.

63. The ring-opened and oligomerized epoxidized palm-based oil composition of claim 57, wherein the composition has about 14.0% wt. to about 18.0% wt. dimer, about 9.0% wt. to about 12.0% wt. trimer, and about 23.0% wt. or greater tetramer and higher order oligomers.

64. The oligomeric polyol of claim 57, wherein the epoxidized palm-based oil composition is made from an oil composition further including an epoxidized non-palm-based oil.

65. The oligomeric polyol of claim 57, wherein the epoxidized non-palm based oil is selected from the group consisting of epoxidized soybean oil, epoxidized safflower oil, epoxidized linseed oil, epoxidized corn oil, epoxidized sunflower oil, epoxidized olive oil, epoxidized canola oil, epoxidized sesame oil, epoxidized cottonseed oil, epoxidized rapeseed oil, epoxidized tung oil, epoxidized fish oil, epoxidized tallow, epoxidized peanut oil, and mixtures thereof.

66. The oligomeric polyol of claim 57, wherein the oligomeric polyol has a Mₙ of about 1000 to 5000 Da.

67. The oligomeric polyol of claim 57, wherein the oligomeric polyol has a M_{w} of about 2000 to 20,000 Da.

68. The oligomeric polyol of claim 57, wherein the oligomeric polyol has a residual epoxy oxygen content of about 0.2% to about 2.5%.

69. The oligomeric polyol of claim 57, wherein the oligomeric polyol is made by reacting epoxidized palm-based oil with a monofunctional alcohol.

70. A method of making an oligomeric polyol comprising the steps of:
(a) providing a fully-epoxidized palm-based oil composition;
(b) providing a ring-opener comprising a monofunctional alcohol; and
(c) reacting the ring-opener with the epoxidized palm-based oil to ring-open and oligomerize the epoxidized palm-based oil to form an oligomeric polyol comprising about 40% wt. or greater oligomers, a hydroxyl number of from about 40 to about 65, a number average hydroxyl functionality of about 2.5 or less, and a viscosity at 25° C. of about 2 Pa·s or less.

71. The method of claim 70, wherein the palm-based oil comprises palm olein.

72. The method of claim 70, wherein the oligomeric polyol has a viscosity at 25° C. of about 1 Pa·s or less.

73. The method of claim 70, wherein the oligomeric polyol has about 50% wt. oligomers or greater.

74. The method of claim 70, wherein the oligomeric polyol has about 14.0% wt. to about 18.0% wt. dimer, about 9.0% wt. to about 12.0% wt. trimer, and about 23.0% wt. or greater tetramer and higher order oligomers.

75. The method of claim 70, wherein the oligomeric polyol has a residual epoxy oxygen content of about 0.2% to about 2.5%.

76. A polyurethane composition comprising the reaction product of:
(a) a polyisocyanate; and
(b) an active hydrogen containing composition comprising the oligomeric polyol of claim 57.

77. The polyurethane composition of claim 76, wherein the active-hydrogen containing composition comprises about 40% to about 90% wt. petroleum-derived polyol and about 10% to about 60% wt. oligomeric polyol.
